SEQUENCE LISTING (12 sequences)

SEQ ID NO: 1 - DNA, 10804 bp, Artificial Sequence
  Chimeric Zika-JEV vrius
  CDS (108)..(10379)
agttgttgatctgtgtgaatcagactgcgacagttcgagtttgaagcgaaagctagcaacagtatcaacaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagttgggaaaaaagaggctatggaaataataaagaagttcaagaaagatctggctgccatgctgagaataatcaatgctaggaaggagaagaagagacgaggaggaaatgaaggctcaatcatgtggctcgcgagcttggcagttgtcatagcttgtgcaggagccatgaagttgtcgaatttccaggggaagcttttgatgaccatcaacaacacggacattgcagacgttatcgtgattcccacctcaaaaggagagaacagatgctgggtccgggcaatcgacgtcggctacatgtgtgaggacactatcacgtacgaatgtcctaagcttaccatgggcaatgatccagaggatgtggattgctggtgtgacaaccaagaagtctacgtccaatatggacggtgcacgcggaccaggcattccaagcgaagcaggagatccgtgtcggtccaaacacatggggagagttcactagtgaataaaaaagaggcttggctggattcaacgaaagccacacgatatctcatgaaaactgagaactggatcataaggaatcctggctatgctttcctggcggcggtacttggctggatgcttggcagtaacaacggtcaacgcgtggtatttaccatcctcctgctgttggtcgctccggcttacagttttaattgtctgggaatgggcaatcgtgacttcatagaaggagccagtggagccacttgggtggacttggtgctagaaggagacagctgcttgacaatcatggcaaacgacaaaccaacattggacgtccgcatgattaacatcgaagctagccaacttgctgaggtcagaagttactgctatcatgcttcagtcactgacatctcgacggtggctcggtgccccacgactggagaagcccacaacgagaagcgagctgatagtagctatgtgtgcaaacaaggcttcactgaccgtgggtggggcaacggatgtggatttttcgggaagggaagcattgacacatgtgcaaaattctcctgcaccagtaaagcgattgggagaacaatccagccagaaaacatcaaatacaaagttggcatttttgtgcatggaaccaccacttcggaaaaccatgggaattattcagcgcaagttggggcgtcccaggcggcagagtttacagtaacacccaatgctccttcggtagccctcaaacttggtgactacggagaagtcacactggactgtgagccaaggagtggactgaacactgaagcgttttacgtcatgaccgtggggtcaaagtcatttctggtccatagggagtggtttcatgacctcgctctcccctggacgtccccttcgagcacagcgtggagaaacagagaactcctcatggaatttgaaggggcgcacgccacaaaacagtccgttgttgctcttgggtcacaggaaggaggcctccatcatgcgttggcaggagccatcgtggtggagtactcaagctcagtgatgttaacatcaggccacctgaaatgtaggctgaaaatggacaaactggctctgaaaggcacaacctatggcatgtgtacagaaaaattctcgttcgcgaaaaatccggtggacactggtcacggaacagttgtcattgaactctcctactctgggagtgatggcccctgcaaaattccgattgtttccgttgcgagcctcaatgacatgacccccgttgggcggctggtgacagtgaaccccttcgtcgcgacttccagtgccaactcaaaggtgctggtcgagatggaaccccccttcggagactcctacatcgtagttggaaggggagacaagcagatcaaccaccattggcacaaagctggaagcacgctgggcaaggccttttcaacaactttgaagggagctcaaagactggcagcgttgggcgacacagcctgggactttggctctattggaggggtcttcaactccataggaagagccgttcaccaagtgtttggtggtgccttcagaacactctttgggggaatgtcttggatcacacaagggctaatgggtgccctactgctctggatgggcgtcaacgcacgagaccgatcaattgctttggccttcttagccacaggaggtgtgctcgtgttcttagcgaccaatgtgcatgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggaccacttctcccttggagtgcttgtgatcctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttactcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaatccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaatgggagttatgttagtgccatcacccaagggaggagggaagaagagactcctgttgagtgcttcgagccctcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaggcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggatcattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagatagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccataatgcttttggggttgctgggaacagtctcgctgggaatcttcttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacctcatacaacaactactccttaatggcgatggccacgcaagctggagtgttgtttggtatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgctgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctagaaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtataacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgtataccaggaggaaggatgtatgcagatgacactgctggctgggacacccgcattagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttagaaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcagatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaagcaccaatcttaatgttgtcaggcctgctagtcagccacagcttggggaaagctgtgcagcctgtgacccccccaggagaagctgggaaaccaagcctatagtcaggccgagaacgccatggcacggaagaagccatgctgcctgtgagcccctcagaggatactgagtcaaaaaaccccacgcgcttggaggcgcaggatgggaaaagaaggtggcgaccttccccacccttcaatctggggcctgaactggagatcagctgtggatccccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatgggtct

SEQ ID NO: 2 - protein, 3422 aa, Artificial Sequence
  Synthetic Construct
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGGNEGSIMWLASLAVVIACAGAMKLSNFQGKLLMTINNTDIADVIVIPTSKGENRCWVRAIDVGYMCEDTITYECPKLTMGNDPEDVDCWCDNQEVYVQYGRCTRTRHSKRSRRSVSVQTHGESSLVNKKEAWLDSTKATRYLMKTENWIIRNPGYAFLAAVLGWMLGSNNGQRVVFTILLLLVAPAYSFNCLGMGNRDFIEGASGATWVDLVLEGDSCLTIMANDKPTLDVRMINIEASQLAEVRSYCYHASVTDISTVARCPTTGEAHNEKRADSSYVCKQGFTDRGWGNGCGFFGKGSIDTCAKFSCTSKAIGRTIQPENIKYKVGIFVHGTTTSENHGNYSAQVGASQAAEFTVTPNAPSVALKLGDYGEVTLDCEPRSGLNTEAFYVMTVGSKSFLVHREWFHDLALPWTSPSSTAWRNRELLMEFEGAHATKQSVVALGSQEGGLHHALAGAIVVEYSSSVMLTSGHLKCRLKMDKLALKGTTYGMCTEKFSFAKNPVDTGHGTVVIELSYSGSDGPCKIPIVSVASLNDMTPVGRLVTVNPFVATSSANSKVLVEMEPPFGDSYIVVGRGDKQINHHWHKAGSTLGKAFSTTLKGAQRLAALGDTAWDFGSIGGVFNSIGRAVHQVFGGAFRTLFGGMSWITQGLMGALLLWMGVNARDRSIALAFLATGGVLVFLATNVHADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPVLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 3 - protein, 104 aa, Zika virus
  MISC_FEATURE (1)..(104): Zika virus N terminal part of C protein
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRR

SEQ ID NO: 4 - protein, 22 aa, Japanese encephalitis virus
  MISC_FEATURE (1)..(22): C terminal signal peptide of C protein
GGNEGSIMWLASLAVVIACAGA

SEQ ID NO: 5 - protein, 666 aa, Japanese encephalitis virus
  MISC_FEATURE (1)..(666): prME protein
MKLSNFQGKLLMTINNTDIADVIVIPTSKGENRCWVRAIDVGYMCEDTITYECPKLTMGNDPEDVDCWCDNQEVYVQYGRCTRTRHSKRSRRSVSVQTHGESSLVNKKEAWLDSTKATRYLMKTENWIIRNPGYAFLAAVLGWMLGSNNGQRVVFTILLLLVAPAYSFNCLGMGNRDFIEGASGATWVDLVLEGDSCLTIMANDKPTLDVRMINIEASQLAEVRSYCYHASVTDISTVARCPTTGEAHNEKRADSSYVCKQGFTDRGWGNGCGFFGKGSIDTCAKFSCTSKAIGRTIQPENIKYKVGIFVHGTTTSENHGNYSAQVGASQAAEFTVTPNAPSVALKLGDYGEVTLDCEPRSGLNTEAFYVMTVGSKSFLVHREWFHDLALPWTSPSSTAWRNRELLMEFEGAHATKQSVVALGSQEGGLHHALAGAIVVEYSSSVMLTSGHLKCRLKMDKLALKGTTYGMCTEKFSFAKNPVDTGHGTVVIELSYSGSDGPCKIPIVSVASLNDMTPVGRLVTVNPFVATSSANSKVLVEMEPPFGDSYIVVGRGDKQINHHWHKAGSTLGKAFSTTLKGAQRLAALGDTAWDFGSIGGVFNSIGRAVHQVFGGAFRTLFGGMSWITQGLMGALLLWMGVNARDRSIALAFLATGGVLVFLATNVH

SEQ ID NO: 6 - protein, 2630 aa, Zika virus
  MISC_FEATURE (1)..(2630): Zika NS proteins
ADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPVLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 7 - protein, 10 aa, Artificial Sequence
  sequence linking Zika C protein and the signal sequence of the C terminal part of the C protein of the Japanese encephalitis virus
EKKRRGGNEG

SEQ ID NO: 8 - protein, 10 aa, Artificial Sequence
  sequence linking JEV prME protein and Zika NS protein
ATNVHADVGC

SEQ ID NO: 9 - protein, 7 aa, Artificial Sequence
  sequence flankeing JEV prME Lys- Glu mutation
QAAEFTV

SEQ ID NO: 10 - protein, 32 aa, Artificial Sequence
  fragment zika virus
RKEKKRRGADTSVGIVGLLLTTAMAAEVTRRG

SEQ ID NO: 11 - protein, 32 aa, Artificial Sequence
  fragment Zika JE chimer
RKEKKRRGADTSVGIVGLLLTTAMAMKLSNFQ

SEQ ID NO: 12 - protein, 35 aa, Artificial Sequence
  fragment Zika JE chimer
RKEKKRRGGNEGSIMWLASLAVVACAGAMKLSNFQ

(19) United States
(12) Patent Application Publication
(10) Pub. No.: US 2022/0168412 A1
(43) Pub. Date: Jun. 2, 2022

(54) Chimeric Zika-Japanese Encephalitis Virus

(71) Applicant: KATHOLIEKE UNIVERSITEIT LEUVEN, Leuven (BE)

(72) Inventors: Kai Dallmeier, Kessel-Lo (BE); Dieudonné Buh Kum, Aarschot (BE); Niraj Mishra, Uttar Pradesh (IN); Johan Neyts, Kessel-Lo (BE)

(21) Appl. No.: 17/602,532

(22) PCT Filed: Apr. 10, 2020
(86) PCT No.: PCT/EP2020/060315

§ 371 Date: Oct. 8, 2021

(30) Foreign Application Priority Data
Apr. 10, 2019 (EP) 19168482.8

(51) Int. Cl. A61K 39/12; A61K 48/00; C12N 15/86; A61P 31/14; A61K 39/00

(52) U.S. Cl. CPC A61K 39/12; A61K 48/00; C12N 15/86; A61K 2039/575; C12N 2770/24171; C12N 2770/24164; C12N 2770/24134; A61P 31/14

(57) ABSTRACT

A polynucleotide comprising the sequence of a live, infectious, attenuated Zika-Japanese encephalitis (JEV) chimeric virus wherein the nucleotide sequence encoding the prME protein of said Zika virus is replaced by a nucleotide sequence encoding the prME protein of a Japanese encephalitis virus, so that said prME protein of said Japanese encephalitis virus is expressed.

### CROSS-REFERENCES TO RELATED APPLICATIONS

This application is a national-stage application under 35 U.S.C. § 371 of International Application No. PCT/EP2020/060315, filed Apr. 10, 2020, which International Application claims benefit of priority to European Patent Application No. 19168482.8, filed Apr. 10, 2019.

### TECHNICAL FIELD

The invention relates to the vaccination of Zika and Japanese encephalitis virus. The invention relates to chimeric viruses of Zika and Japanese encephalitis virus.

### BACKGROUND

In spite of its spread worldwide, flaviviruses are mostly restricted to their defined endemic geographical areas. However, recent trends suggest that flavivirus infections are not only escalating in magnitudes, but also are spreading to new areas and, therefore, flaviviruses are classified as emerging and re-emerging viruses [Ishikawa et al. (2014) Vaccine. 32, 1326-1337; Mackenzie et al. (2004) Nat Med. 10(12S), S98-109; Petersen et al. (2016) N Engl J Med. 374(16), 1552-1563; Wilder-Smith & Monath (2016) Lancet Infect Dis. 22. 1473-3099(16), 30588-30586].

c-LAV is defined as a Chimeric infectious Live-Attenuated Virus (e.g. YFV 17D), in which the nucleotide sequence of a prM-E protein is replaced with other flaviviruses (e.g. Japanese encephalitis virus, dengue, Zika etc.), so that functional prM-E protein, which is expressed in chimera does not belong to parental flavivirus. In spite of several safety issues, LAVs are most efficient vaccines as it provides both humoral and cellular immunity [Monath et al. (2015) Vaccine 33, 62-72; Minor (2015) Virol. 479-480, 379-392].

It is widely accepted in the art that all the humoral responses against flaviviruses are mainly because of domain 3 loop of its envelope protein, which results in the genesis of neutralizing antibodies [Li et al. (2014) Hum Vaccin Immunother. 10, 3579-3593].

To date, the vaccine field broadly relies on the strategy to mount protective immunity against structural proteins of a virus, in case of Flaviviruses in particular neutralizing antibodies (nAb) elicited against the viral surface proteins (WHO Expert Committee on Biological Standardisation, WHO Technical Report Series 979, 2011, Annex 2: Guidelines on the quality, safety and efficacy of dengue tetravalent vaccines (live, attenuated); WHO Expert Committee on Biological Standardisation, Recommendations to assure the quality, safety and efficacy of Japanese encephalitis vaccines (live, attenuated) for human use, Technical Report Series 980, 2012). For instance, two vaccines against JEV are approved, namely Ixiaro® (an inactivated vaccine) and Imojev® (a YFV 17D-based chimeric live-attenuated vaccine comprising prM protein and envelope E protein of Japanese encephalitis virus; c-LAV) [Li et al. (2014) Hum Vaccin Immunother. 10, 3579-3593; Scott (2016) Drugs 76, 1301-1312] based on their ability to induce sufficiently high serum titres of JEV-specific nAb that are widely accepted as immune correlates of protection.

Chimeric vaccines have been developed wherein the prME genes of the envisaged pathogen are inserted into the backbone of another flavivirus.

Mathenge et al. (2004) J. Gen. Virol. 85, 2503-2513 and Yang (2017) Protein Cell 8, 225-229 disclose dual vaccination with chimeric viruses with a Japanese encephalitis virus backbone and Dengue Prm-E proteins.

The Imojev Vaccine against Japanese encephalitis (JE), is a yellow fever virus wherein the YF prME genes of the well-established YF17D yellow fever vaccine are replaced by those of the JE virus. The safety of the YFD vaccine has been the reason to consider this construct as a backbone for chimeric viruses. Unintentionally, the chimeric vaccine contains fragments of geographically distinct regions. Thus the unmodified yellow fever vaccine for vaccination of yellow fever is used in other geographic regions (Africa and South America) than the chimeric vaccine for Japanese encephalitis (Asia).

The recent outbreak of ZIKA in the Americas has led to the search of therapeutics to curb or prevent the debilitating disease. As of now, a number of antivirals and vaccine candidates have been reported that show activity against the ZIKA virus (ZIKV) but none has been approved yet even during an emergency. The Japanese encephalitis (JEV) and ZIKA viruses belong to a genus called Flavivirus together with the Yellow Fever, Dengue, West Nile, Tick borne encephalitis viruses.

Vaccines against flaviviruses have been developed, such as the well-established attenuated YF17D yellow fever vaccine.

Furthermore chimeric vaccines have been developed wherein the prM/E genes of one flavivirus are inserted, or replaced, in the prM/E genes of another flavivirus. The best known examples hereof are the Chimerivax viruses (Guirakhoo et al. (1999) Virology 257, 363-372).

It has been found that the C terminal part of the C protein acts a signal peptide and it important in the proper proteolytic processing of the viral proteins. To what extent a chimeric construct should contain the signal sequence of the parent virus or from the insert is unpredictable, since optimal processing and viral fitness are not correlated (see Lee et al. (2000) J. Virol. 74, 24-32 and Lobigs & Lee (2004) J. Virol. 78, 178-86).

The Japanese encephalitis virus (JEV) and Zika virus (ZIKV) are flaviviruses of emerging public health concern. Generally, neutralizing antibodies (nAb) that target the flavivirus envelope are considered to correlate with the protection against infection with flaviviruses. For JEV, there are two nAb inducing vaccines commercially available, (a) an inactivated vaccine (Ixiaro®) and (b) a chimeric live-attenuated vaccine (Imojev®, approved in Australia and Thailand only). However, till date no vaccine is approved against ZIKV. Various ZIKV nAb inducing Zika vaccine candidates are in different phases of (pre-) clinical development. Growing evidences suggests that also non-structural proteins of flaviviruses can serve as protective antigens.

There is no approved ZIKV vaccine available. Live-attenuated vaccines are the most efficient entities among different vaccine classes, ideally requiring only one dose to induce long-lasting immunity. Prequalified live-attenuated vaccines are not globally available against ZIKV and JEV. The chimeric live-attenuated JEV vaccine (Imojev®) needs to be administered 2 time for efficient vaccination. Various ZIKV envelop based Zika vaccine candidates are in different phases of (pre)clinical development. However, neutralizing antibodies (nAb) targeted against the ZIKV envelope have been shown to cross-react with dengue virus envelope protein potentially causing antibody-dependent enhancement (ADE) of dengue disease.

### SUMMARY

The present invention is based on the finding that the C protein and the non-structural proteins of a chimeric virus are sufficient to act as a vaccine, such that a chimeric vaccines not only function against the virus from which the prME insert originates, but also against the virus from which the remaining backbone portion originates.

The invention thus relates to chimeric live-attenuated Flaviviruses of the general genetic structure A-B-A and their use in vaccination against at least A, wherein A is particular virus A originally used as vector backbone (vector) and (ii) B representing the genetic material of another virus B and encoding for antigenic surface proteins of B (vaccine target) that has originally been introduced into A for the purpose of inducing protection from said virus B infection following vaccination. Surprisingly such A-B-A vaccines can also been used to efficiently immunize against and fully protect from virus A (e.g. YFV in the case of Imojev® and Dengvaxia®) that was originally considered as antigenically irrelevant and employed only as vector component. The underlying mechanism conferring protection from A is specific for A, yet unrelated to that of the original A-B-A vaccine as it does not involve neutralizing antibodies.

To develop a dual JEV-ZIKV vaccine avoiding the risk of ADE, we generated a chimeric live-attenuated ZIK-JEprM/E vaccine by replacing the prM/E genes within the backbone of a ZIKV clone with that of the JEV. This intervention results in live-attenuated vaccine which provide protection against JEV by induction of envelop based nAbs against JEV and against ZIKV by induction of immunity against ZIKV non-structural proteins (based non-nAbs and cellular immunity). As ZIK-JEprM/E fails to induce antibodies against ZIKV envelop, ADE-mediated dengue virus infection is less likely.

The invention discloses an attenuated version of a originally pathogenic ZIKV strain which has the advantage that can it be used as live-attenuated vaccine, as dual vaccine, and with lower risk of consecutive severe dengue virus infection due to antibody dependent enhancement.

Non-structural proteins, especially NS1 due to antigen presentation and secretory nature can also be important in cellular and humoral responses. Therefore, it appears that humoral and cellular responses against both envelope and non-structural proteins may form the basis of vaccination [Pierson et al. (2008) Cell Host Microbe. 4, 229-238; Putnak et al. (1990) J Gen Virol. 71(Pt 8), 1697-1702; Schlesinger et al. (1993) Virology. 92(1), 132-141; Amorim et al. (2016) Virology 487, 41-49; Rastogi et al. (2016) Virol J. 13, 131; Watterson et al. (2016) Antiviral Res. 130, 7-18.]. c-LAV flavivirus vaccines (e.g. Imojev or Dengvaxia®) contain a structural part from one virus (e.g. JEV or Dengue) and a non-structural part from other virus (e.g. YFV) and therefore, they have a potential to be developed as a dual vaccine against two flaviviruses.

The present invention demonstrates that in a chimeric virus, the presence of the non-structural parental antigens, even in the absence of the parental PrME gene, is sufficient to be used as a vaccine against the parental virus.

The present invention further has the advantages that vaccination can be performed against a virus infection associated with antibody dependent enhancement, such as is the case with Zika.

Indeed, whereas a first infection generally results in a mild disease, a second infection generally provokes a more severe disease pattern.

The fact that subsequent infections are more severe can be attributed to the development of Antibody Dependent Enhancement (ADE). The antibodies responsible for this phenomenon are raised against the Envelope protein, and especially against the prM protein.

Upon use of a chimeric flavivirus vaccine, antibodies are raised against the Capsid proteins and the Non-structural proteins of the backbone and against the membrane protein and the envelope protein. It is generally accepted in the field that the antibodies against the membrane and/or envelope protein are essential to provide protection against the pathogen.

Although the skilled person recognized that the Non-structural proteins encoded by the backbone will also generate antibodies, this response is not believed to be sufficient to protect an individual from a viral infection. As a consequence chimeric vectors comprise the prM and E genes of the envisaged pathogen, which may lead to antibody-dependent enhancement (ADE), especially in the case of Dengue or Zika.

It an aspect of the invention to provide a vaccination where the involvement of neutralizing antibodies originating from the envelope protein or the membrane protein is circumvented.

The invention is further summarized in the following statements:

1. A polynucleotide comprising the sequence of a live, infectious, attenuated Zika-Japanese encephalitis (JEV) chimeric virus wherein the nucleotide sequence encoding the prME protein of said Zika virus is replaced by a nucleotide sequence encoding the prME protein of a Japanese encephalitis virus, so that said prME protein of said Japanese encephalitis virus is expressed.

Thus the nucleotide sequence encodes the prME protein a Japanese encephalitis virus, so that said prME protein of said Japanese encephalitis virus is expressed, and the nucleotide sequence encodes the signal sequence of the C terminal part of the C protein of the Japanese encephalitis virus so that said signal sequence of said Japanese encephalitis virus is expressed.

2. The polynucleotide according to statement 1, wherein further the nucleotide sequence encoding the signal sequence of the C terminal part of the C protein of the Zika virus is replaced by a nucleotide sequence encoding the signal sequence of the C terminal part of the C protein of the Japanese encephalitis virus so that said signal sequence of said Japanese encephalitis virus is expressed.

Thus, the nucleotide sequence encodes the signal sequence of the C terminal part of the C protein of the Japanese encephalitis virus so that said signal sequence of said Japanese encephalitis virus is expressed.

3. The polynucleotide according to statement 1 or 2, which further differs from the wild type sequence by an A to G nucleotide substitution at position 1482 as depicted in SEQ ID NO:1.

4. The polynucleotide according to any one of statement 1 to 3, wherein the encoded amino acid sequence of the JEV prME protein has the sequence of SEQ ID NO:5.

5. The polynucleotide according to any one of statements 1 to 4, wherein the encoded amino acid sequence of the signal sequence of the C terminal part of the C protein of the Japanese encephalitis virus has the sequence GGNEGSIMWLASLAVV [SEQ ID NO:4].

6. The polynucleotide according to any one of statements 1 to 5, wherein the encoded amino acid sequence linking the Zika C protein and the signal sequence of the C terminal part of the C protein of the Japanese encephalitis virus comprise the sequence EKKRR GGNEG [SEQ ID NO:7]

7. The polynucleotide according to any one of statements 1 to 6, wherein the encoded amino acid sequence linking JEV prME protein and the Zika NS protein comprises the sequence ATNVH ADVGC [SEQ ID NO:8].

8. The polynucleotide according to any one of statements 1 to 7, wherein the Zika virus is the BeH819015 strain.

9. The polynucleotide according to any one of statements 1 to 8, comprising the open reading frame from nucleotide 108 to 10379 depicted in SEQ ID NO:1. (stopcodon included).

10. The polynucleotide according to any one of statements 1 to 9, comprising the nucleotide sequence depicted in SEQ ID NO:1.

11. The polynucleotide according to any one of statements 1 to 10, encoding the polypeptide sequence depicted in SEQ ID NO:2.

12. The polynucleotide according to any one of statements 1 to 11, which is an Artificial Bacterial Chromosome.

13. The polynucleotide according to statement 12, wherein the BAC comprises:
an inducible bacterial ori sequence for amplification of said BAC to more than 10 copies per bacterial cell, and
a viral expression cassette comprising a cDNA of said zika-JEV chimeric virus and comprising cis-regulatory elements for transcription of said viral cDNA in mammalian cells and for processing of the transcribed RNA into infectious RNA virus.

14. A live, infectious, attenuated Zika-Japanese encephalitis (JEV) chimeric virus wherein the prME protein of said Zika virus is replaced by the prME protein of a Japanese encephalitis virus.

15. The Zika-JEV chimeric virus according to statement 14, wherein further the signal sequence of the C terminal part of the C protein of said Zika virus is replaced by the signal sequence of the C terminal part of the C protein of a Japanese encephalitis virus.

16. The Zika-JEV chimeric virus according to statement 14 to 15 comprising in the prME polypeptide the sequence QAAEFTV [SEQ ID NO:9].

17. The Zika-JEV chimeric virus according to any one of statements 14 to 16, wherein the signal sequence and the prME protein of the JEV virus has the amino acid sequence depicted in SEQ ID 4 and 5.

18. The Zika-JEV chimeric virus according to any one of statements 14 to 17, wherein the JEV virus is SA14-14-2.

19. The Zika-JEV chimeric virus according to any one of statements 14 to 18, wherein the Zika virus is the BeH819015 strain.

20. The Zika-JEV chimeric virus according to any one of statements 14 to 19, comprising the amino acid sequence GGNEGSIMWLASLAVV [SEQ ID NO:4].

21. The Zika-JEV chimeric virus according to any one of statements 14 to 20, wherein the amino acid sequence at the junction of the C protein of the Zika virus and the signal peptide of the JEV virus has the amino acid sequence EKKRR GGNEG [SEQ ID NO:7].

22. A Zika Japanese encephalitis chimeric virus according to any one of statements 14 to 21, for use as a vaccine.

23. A Zika Japanese encephalitis chimeric virus according to statement 22, for use as a vaccine in the prevention against Zika virus and/or Japanese encephalitis.

24. The Zika Japanese encephalitis chimeric virus according to statement 22, for use as a vaccine in the simultaneous prevention against Zika virus and Japanese encephalitis.

25. The polynucleotide according any one of statements 1 to 13 encoding a Zika Japanese encephalitis chimeric virus, for use as a vaccine.

26. The polynucleotide according to statement 25 for use as a vaccine in the prevention against Zika virus and/or Japanese encephalitis.

27. The polynucleotide according to statement 25 for use as a vaccine in the simultaneous prevention against Zika virus and Japanese encephalitis.

28. A pharmaceutical composition comprising a polynucleotide sequence encoding a Zika Japanese encephalitis chimeric virus according any one of statements 1 to 13, and a pharmaceutical acceptable carrier.

29. A pharmaceutical composition comprising a Zika Japanese encephalitis chimeric virus according any one of statements 14 to 21, and a pharmaceutical acceptable carrier.

30. A method of preparing a vaccine against a Zika and/or Japanese encephalitis infection, comprising the steps of:
a) providing a BAC which comprises
   an inducible bacterial ori sequence for amplification of said BAC to more than 10 copies per bacterial cell, and
a viral expression cassette comprising a cDNA of a zika_JEV chimeric virus according to any one of statements 1 to 13, and comprising cis-regulatory elements for transcription of said viral cDNA in mammalian cells and for processing of the transcribed RNA into infectious RNA virus
b) transfecting mammalian cells with the BAC of step a) and passaging the infected cells
c) validating replicated virus of the transfected cells of step b) for virulence and the capacity of generating antibodies and inducing protection against Zika virus infection,
d) cloning the virus validated in step c into a vector,
e) formulating the vector into a vaccine formulation.

31. The method according to statement 30, wherein the vector is a BAC which comprises an inducible bacterial ori sequence for amplification of said BAC to more than 10 copies per bacterial cell.

32. A method of inducing a neutralizing and/or protective antibody response against JE virus and/or Zika virus in a subject, thereby preventing an infection by JE virus and/or Zika, comprising the step of administering to said subject the Zika-Japanese encephalitis chimeric virus of statements 14 to 21.

33. A method of inducing a protective immune response against JE virus and/or Zika virus in a subject, thereby preventing an infection by JE virus and/or Zika, comprising the step of administering to said subject the Zika-Japanese encephalitis chimeric virus of claims 14 to 21.

32. A method of inducing a protective immune response against JE virus and/or Zika virus in a subject, thereby preventing an infection by JE virus and/or Zika, yet avoiding the induction of antibodies that enhance subsequent dengue and/or Zika infection by the mechanism of antibody-dependent enhancement (ADE), comprising the step of administering to said subject the Zika-Japanese encephalitis chimeric virus of claims 14 to 21.

35. A method of inducing a protective immune responses against flaviviruses from different serocomplexes such as the Japanese encephalitis virus and the Zika virus in a subject, thereby preventing an infection by either virus, yet avoiding the induction of antibodies that enhance subsequent dengue and/or Zika infection by the mechanism of antibody-dependent enhancement (ADE), comprising the step of administering to said subject the a chimeric flavivirus.

The administration thus results in one or more of inducing immunity to JE, inducing immunity to Zika, inducing antibodies against JE, inducing virus-neutralising antibodies against, inducing cellular immunity against JE, inducing antibodies against ZIK, inducing cellular immunity against ZIK.

### BRIEF DESCRIPTION OF THE DRAWINGS

FIG. 1: Amino acid sequences of the capsid-prM (C-prM) junctions in wild-type and chimeric ZIKV used in the present study. Amino acid sequences upstream and downstream of the C anchor domain (=prM signal peptide) are shown in single letter SI abbreviation; (2b/3)—NS2b/3 protease cleavage site; (sign)—signal peptidase cleavage site; *—gap introduced to facilitate sequence alignments.

FIGS. 2A-2E: Construction and propagation of wild-type and chimeric viruses. FIG. 2A is a schematic representation of wild-type ZIKV and JEV and their chimeric derivatives. Chimeric viruses were constructed by swapping the prM/E of ZIKV (with or without the capsid anchor sequence) with corresponding sequences of the live attenuated JEV SA 14-14-2. FIG. 2B shows immunostaining of ZIKV and chimeric viruses on Vero E6 cells. Cells were transfected with plasmids containing either wild-type ZIKV, ZIK-JEprM/E with ZIKV Canch (ZIK-JEprM/E ZIKCanch) or JEV Canch (ZIK-JEprM/E JECanch). One day post transfection (dpt), cells were trypsinized and parts transferred to 8-well chambered slides for immunohistostaining with a monoclonal pan flavivirus monoclonal Ab (mAb) 4G2 at days 3 and 5 dpt. FIG. 2C shows antigenicity of ZIK-JEprM/E. Vero E6 cells were infected with either ZIKV, JEV or ZIK-JEprM/E JECanch at MOI of 0.1 and stained 2 days post infection (dpi) with mAb 4G2, a ZIKV E-specific mAb, a JEV E-specific mAb, and a ZIKV NS1-specific mAb. FIG. 2D shows replication kinetics of ZIKV (closed circles) and ZIK-JEprM/E (close squares) on BHK-21J cells. BHK-21J cells were seeded overnight in a 6-well plate and infected with either ZIKV or ZIK-JEprM/E at a MOI of 0.01 and the extracellular infectious virus was quantified daily by plaque assay. FIG. 2E shows plaque size of ZIKV and ZIK-JEprM/E on BHK-21J cells developed and stained 6 dpi. Data are presented as mean values with error bars indicating SEM of n=2 replicates. Dotted line denotes the limit of detection (L.O.D) of the assay.

FIG. 3 Interferon gamma (IFN-γ) ELISPOT following stimulation of splenocytes from vaccinated and sham-vaccinated mice with indicated antigens. Interferon alpha/beta (IFN-α/β)-deficient mice were either vaccinated intraperitoneally (i.p.) with 104 PFU of ZIK-JEprM/E (n=5) or sham-vaccinated (using with 2% FBS medium; n=3). Ten weeks post vaccination, splenocytes were harvested and subjected to IFN-γ ELISPOT. Data are presented as mean values with error bars indicating SEM. Values are normalized by subtracting the number of spots in control wells (stimulated with Vero E6 cell lysate only).

FIGS. 4A and 4B: Protective efficacy of ZIK-JEprM/E against lethal JEV and ZIKV infections in AG129. Mice were either vaccinated with 104 PFU of ZIK-JEprM/E (n=8), chimeric virus YF-JEprM/E (n=8) or non-vaccinated with 2% FBS medium (n=8). Twenty-eight days post vaccination, both vaccinated and non-vaccinated mice were i.p. challenged with 1000 LD50 of JEV. (FIG. 4A) Survival curves of vaccinated and non-vaccinated mice observed for 35 days after JEV challenge. (FIG. 4B) Survivals in (FIG. 4A) i.e. ZIK-JEprM/E vaccinated (n=8), YF-JEprM/E (n=7) were later i.p. challenged with 104 PFU of ZIKV and further observed for 28 days. A group of non-vaccinated mice (n=6) was added as control in the ZIKV challenge.

FIG. 5 shows the nucleotide sequence of vaccine ZIK-JEprM/E with nucleotides derived from ZIKV (strain BeH819015) in, nucleotides derived from JEV (vaccine strain SA14-14-2) i and the JECanch cDNA sequence; A1482G mutation arising in the JEV-E gene region by passaging on Vero E6 is indicated.

### DETAILED DESCRIPTION

Flaviviruses belong to the viral family of Flaviviridae and comprise many medically important viruses, including Dengue virus (DENV), Japanese encephalitis virus (JEV), West Nile virus (WNV), Tick-borne encephalitis virus (TBEV), Yellow fever virus (YFV) and Zika virus (ZIKV), Japanese Encephalitis virus (JEV), Murray Valley Encephalitis virus (MVE), St. Louis Encephalitis virus (SLE), Tick-borne Encephalitis (TBE) virus, Russian Spring-Summer Encephalitis virus (RSSE), Kunjin virus, Powassan virus, Kyasanur Forest Disease virus, Usutu virus, Wesselsbron and Omsk Hemorrhagic Fever virus. Flaviviruses are enveloped with a ˜10-11 kb long (+)ssRNA genome encoding for 3 structural proteins (core, C; premembrane, prM; and envelope, E), which are incorporated in the virions, and 7 nonstructural proteins (NS1, NS2A, NS2B, NS3, NS4A, NS4B and NS5).

The signal peptide at the C terminus of the C protein (C-signal peptide; also called C-anchor domain (“canch”) regulates flavivirus packaging through coordination of sequential cleavages at the N terminus (by viral NS2B/NS3 protease in the cytoplasm) and C terminus (by host signalase in the endoplasmic reticulum [ER] lumen) of the signal peptide sequence.

Generally, a c-LAV (Chimeric Life Attenuated Vaccine) of the prior art is defined as an infectious live-attenuated vaccine virus in which the nucleotide sequences of the structural prM and E proteins are replaced with those of another Flavivirus (e.g. JEV, DENY, ZIKV etc.). The generic structure of such c-LAV can be described as A-B-A with (i) building blocks A comprise parts of a particular virus A originally used as vector backbone [typically the encoding the C protein and the Ns proteins](vector) and (ii) building blocks B representing the genetic material of another virus B and encoding for the antigenic surface proteins of B (vaccine target) that has originally been introduced into A for the purpose of inducing protection from said virus B infection following vaccination.

Following the same A-B-A blueprint, other DENY c-LAVs are currently in development, yet using attenuated DENY strains (Torresi et al. (2017) Hum Vaccin Immunother. 13, 1059-1072) or ZIKV (Xie et al (2017) MBio. 8, e02134-16) as vector backbone. Similar c-LAV candidates has been developed for a variety of Flaviviruses each time targeting for neutraling antibody (nAb) responses by the envelope and membrane protein (Lai et al. (2003) Adv Virus Res. 61, 469-509) including DENV4/TBEV (Langatvirus) chimeras to protect from TBEV by nAb neutralizing TBEV (Pletnev & Men (1998) Proc Natl Acad Sci USA. 95, 1746-1751).

Prior vaccine strategies focused on the induction of nAb responses that protect from Flavivirus infections and are mostly directed against the respective envelope protein [Li et al. (2014) Hum Vaccin Immunother. 10, 3579-3593]. However, in addition to this widely accepted correlate of vaccine-mediated protection by nAb, historical as well as recently published evidence suggested that, in addition, nonstructural proteins and, due to its antigenic and secreted nature, in particular NS1 may contribute to some extent to vaccine efficacy via protective cellular and humoral immune responses [Pierson et al. (2008) Cell Host Microbe. 4, 229-238; Putnak et al. (1990) J Gen Virol. 71(Pt 8), 1697-1702; Schlesinger et al. (1993) Virology 92(1), 132-141; Amorim et al. (2016) Virology 487, 41-49; Rastogi et al. (2016) Virol J. 13, 131; Watterson et al. (2016) Antiviral Res. 130, 7-18]. We here show that, unexpectedly, a c-LAV Flavivirus vaccine approved to protect against JEV has the potential to fully protect from massively lethal vaccine challenge fully without induction of any relevant nAb and may therefore be developed as dual vaccines, also protecting against a second Flavivirus, namely against Zika virus which provided the backbone of non-structural proteins for replication of the c-LAV. In other words, c-LAVs of the generic structure A-B-A protect against infection with two viruses B and A because these chimeric vaccines contain the structural proteins of one virus B (JE) and the nonstructural proteins of a second virus A (Zika).

A BAC as referred to in the present application comprises:
an inducible bacterial ori sequence for amplification of said BAC to more than 10 copies per bacterial cell, and
a viral expression cassette comprising a cDNA of an the RNA virus genome and comprising cis-regulatory elements for transcription of said viral cDNA in mammalian cells and for processing of the transcribed RNA into infectious RNA virus.

As is the case in the present invention the RNA virus genome is a chimeric viral cDNA construct of two RNA virus genomes.

In these BACS, the viral expression cassette comprises a cDNA of a positive-strand RNA virus genome, an typically
a RNA polymerase driven promoter preceding the 5′ end of said cDNA for initiating the transcription of said cDNA, and
an element for RNA self-cleaving following the 3′ end of said cDNA for cleaving the RNA transcript of said viral cDNA at a set position.

The BAC may further comprise a yeast autonomously replicating sequence for shuttling to and maintaining said bacterial artificial chromosome in yeast. An example of a yeast ori sequence is the 2μ plasmid origin or the ARS1 (autonomously replicating sequence 1) or functionally homologous derivatives thereof.

The RNA polymerase driven promoter of this first aspect of the invention can be an RNA polymerase II promoter, such as Cytomegalovirus Immediate Early (CMV-IE) promoter, or the Simian virus 40 promoter or functionally homologous derivatives thereof.

The RNA polymerase driven promoter can equally be an RNA polymerase I or III promoter.

The BAC may also comprise an element for RNA self-cleaving such as the cDNA of the genomic ribozyme of hepatitis delta virus or functionally homologous RNA elements.

The formulation of DNA into a vaccine preparation is known in the art and is described in detail in for example chapter 6 to 10 of “DNA Vaccines” Methods in Molecular Medicine Vol 127, (2006) Springer Saltzman, Shen and Brandsma (Eds.) Humana Press. Totoma, N.J. and in chapter 61 Alternative vaccine delivery methods, Pages 1200-1231, of Vaccines (6th Edition) (2013) (Plotkin et al. Eds.). Details on acceptable carrier, diluents, excipient and adjuvant suitable in the preparation of DNA vaccines can also be found in WO2005042014, as indicated below.

“Acceptable carrier, diluent or excipient” refers to an additional substance that is acceptable for use in human and/or veterinary medicine, with particular regard to immunotherapy.

By way of example, an acceptable carrier, diluent or excipient may be a solid or liquid filler, diluent or encapsulating substance that may be safely used in systemic or topic administration. Depending upon the particular route of administration, a variety of carriers, well known in the art may be used. These carriers may be selected from a group including sugars, starches, cellulose and its derivatives, malt, gelatine, talc, calcium sulphate and carbonates, vegetable oils, synthetic oils, polyols, alginic acid, phosphate buffered solutions, emulsifiers, isotonic saline and salts such as mineral acid salts including hydrochlorides, bromides and sulphates, organic acids such as acetates, propionates and malonates and pyrogen-free water.

A useful reference describing pharmaceutically acceptable carriers, diluents and excipients is Remington's Pharmaceutical Sciences (Mack Publishing Co. N. J. USA, 1991) which is incorporated herein by reference.

Any safe route of administration may be employed for providing a patient with the DNA vaccine. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Intra-muscular and subcutaneous injection may be appropriate, for example, for administration of immunotherapeutic compositions, proteinaceous vaccines and nucleic acid vaccines. It is also contemplated that microparticle bombardment or electroporation may be particularly useful for delivery of nucleic acid vaccines.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

DNA vaccines suitable for oral or parenteral administration may be presented as discrete units such as capsules, sachets or tablets each containing a pre-determined amount of plasmid DNA, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the DNA plasmids with liquid carriers or finely divided solid carriers or both, and then, if necessary, shaping the product into the desired presentation.

The above compositions may be administered in a manner compatible with the dosage formulation, and in such amount as is effective. The dose administered to a patient, should be sufficient to effect a beneficial response in a patient over an appropriate period of time. The quantity of agent (s) to be administered may depend on the subject to be treated inclusive of the age, sex, weight and general health condition thereof, factors that will depend on the judgement of the practitioner.

Furthermore DNA vaccine may be delivered by bacterial transduction as using live-attenuated strain of Salmonella transformed with said DNA plasmids as exemplified by Darji et al. (2000) FEMS Immunol Med Microbiol 27, 341-349 and Cicin-Sain et al. (2003) J. Virol. 77, 8249-8255 given as reference.

Typically the DNA vaccines are used for prophylactic or therapeutic immunisation of humans, but can for certain viruses also be applied on vertebrate animals (typically mammals, birds and fish) including domestic animals such as livestock and companion animals. The vaccination is envisaged of animals which are a live reservoir of viruses (zoonosis) such as monkeys, mice, rats, birds and bats.

In certain embodiments vaccines may include an adjuvant, i.e. one or more substances that enhances the immunogenicity and/or efficacy of a vaccine composition However, life vaccines may eventually be harmed by adjuvants that may stimulate innate immune response independent of viral replication. Non-limiting examples of suitable adjuvants include squalane and squalene (or other oils of animal origin); block copolymers; detergents such as Tween-80; Quill A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); interleukins such as interleukin 2 and interleukin 12; monokines such as interleukin 1; tumour necrosis factor; interferons such as gamma interferon; combinations such as saponin-aluminium hydroxide or Quil-A aluminium hydroxide; liposomes; ISCOMt) and ISCOMATRIX (B) adjuvant; mycobacterial cell wall extract; synthetic glycopeptides such as muramyl dipeptides or other derivatives; Avridine; Lipid A derivatives; dextran sulfate; DEAE-Dextran or with aluminium phosphate; carboxypolymethylene such as Carbopol'EMA; acrylic copolymer emulsions such as Neocryl A640; vaccinia or animal poxvirus proteins; sub-viral particle adjuvants such as cholera toxin, or mixtures thereof.

### EXAMPLES

### Example 1

### Construction of ZIK-JEprM/E

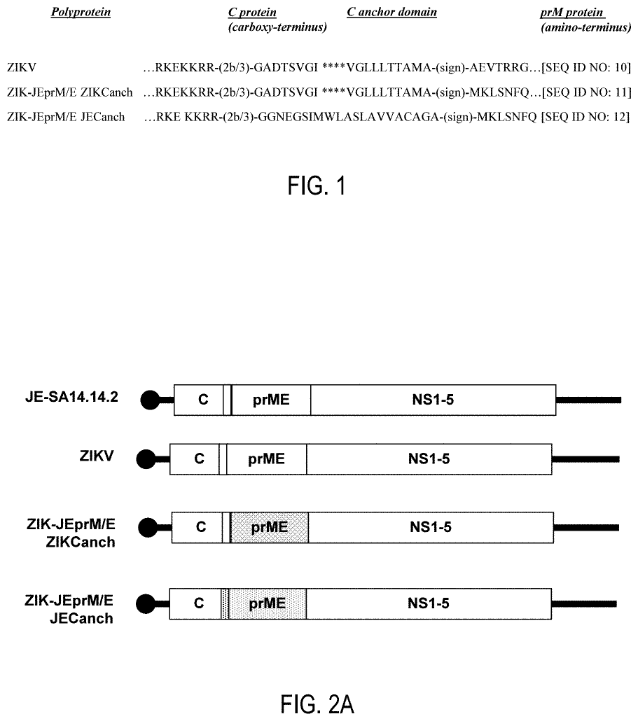

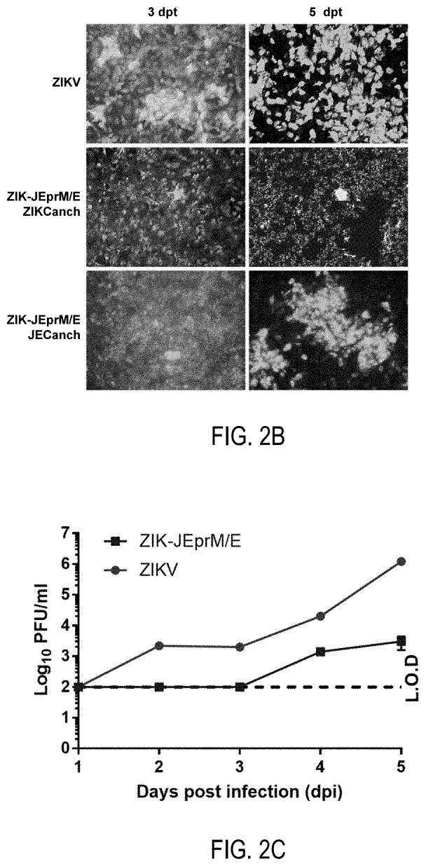

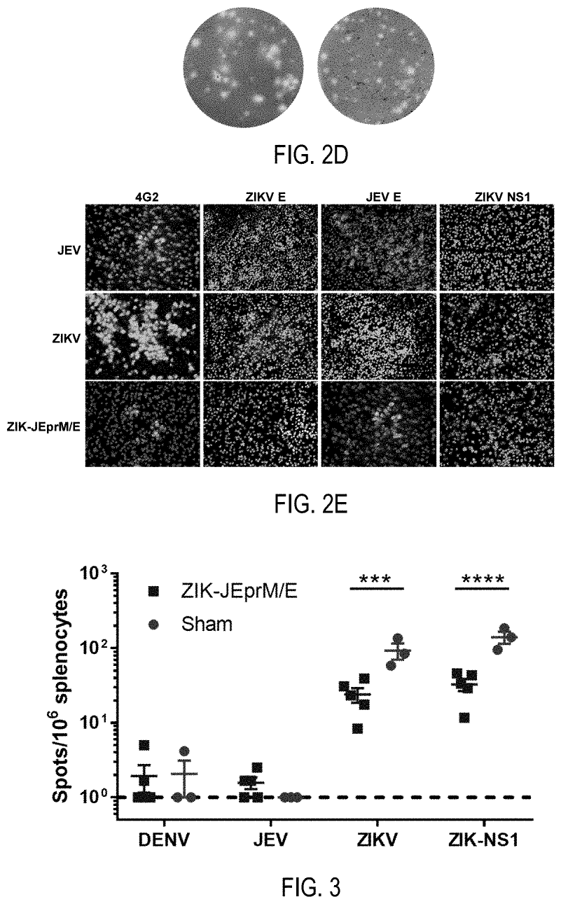

The ZIKV strain BeH819015 cDNA was cloned into pShuttleBAC according to WO2014174078A1 to yield pShuttle-ZIKV. A chimeric replication competent flavivirus vaccine was generated by exchanging the prM/E coding sequence of the ZIKV in pShuttle-ZIKV for the respective JEV sequence (vaccine strain SA14-14-2) using standard recombinant DNA techniques and recombination in yeast. Two variants pShuttle-ZIK-JEprM/E_JECanch and pShuttle-ZIK-JEprM/E_ZIKCanch were generated containing either the ZIKV or JEV derived Canch domains (FIGS. 1 and 2A).

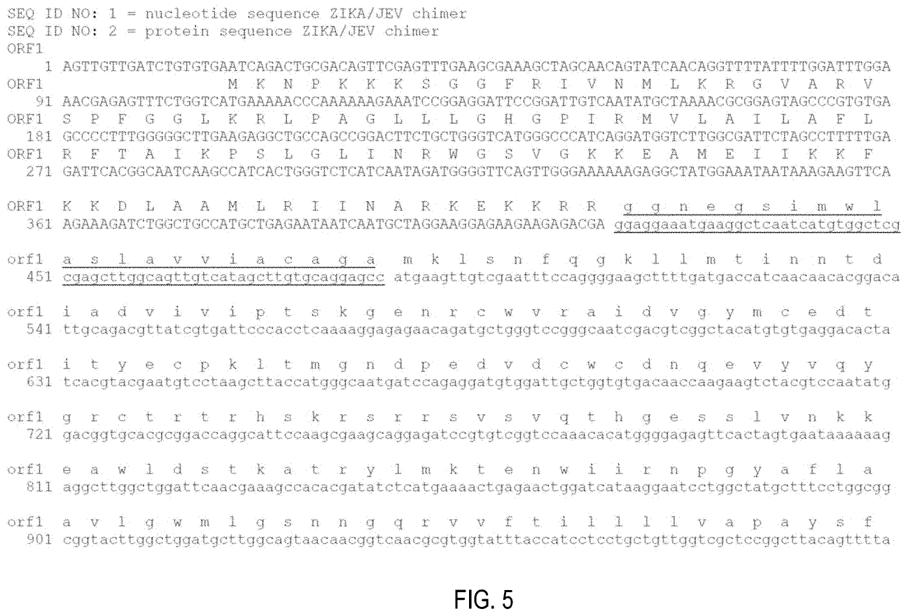

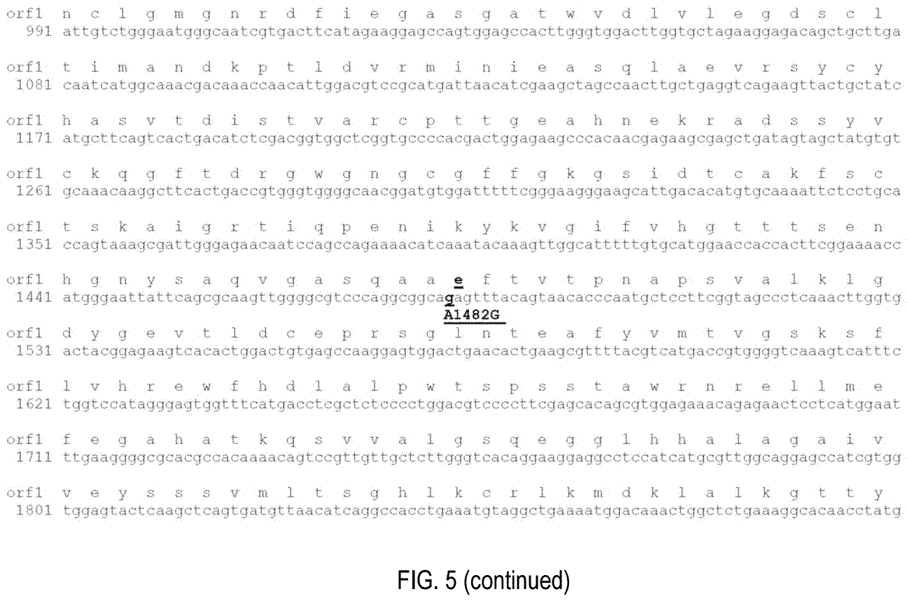

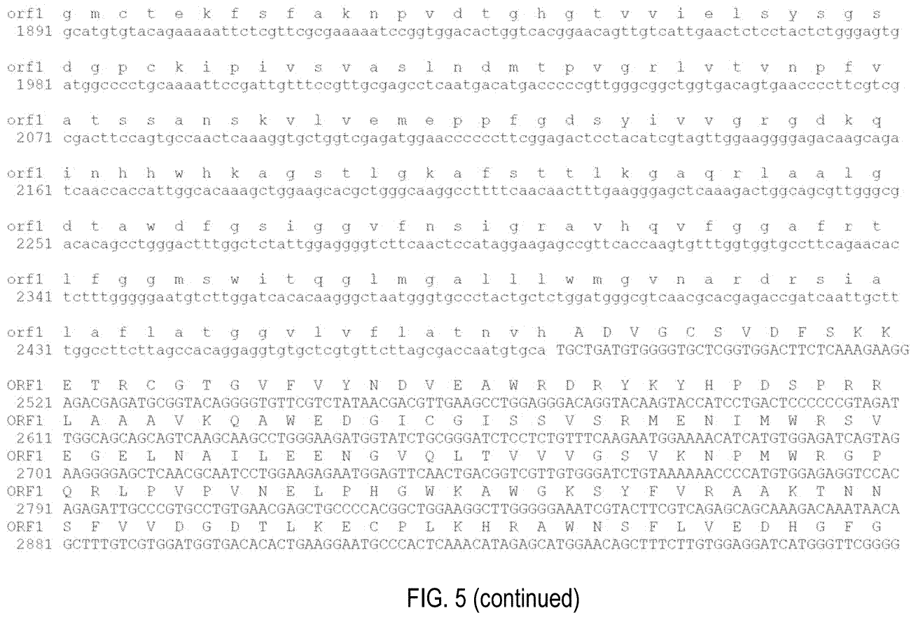

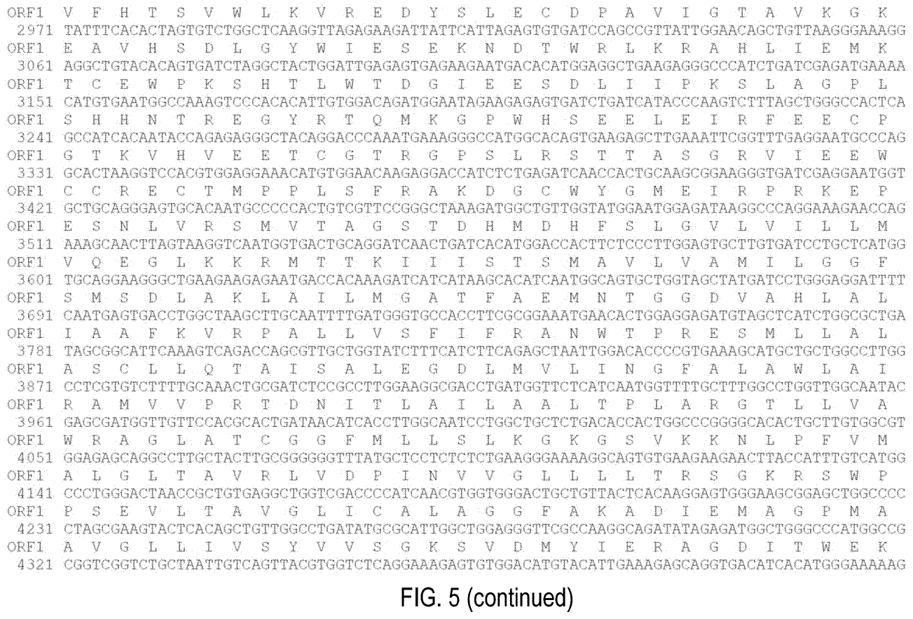

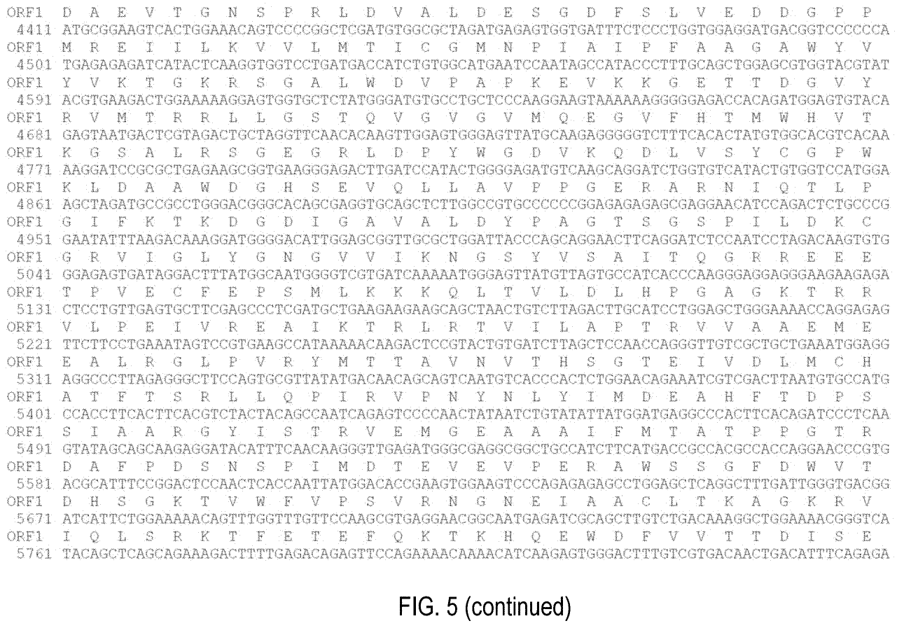

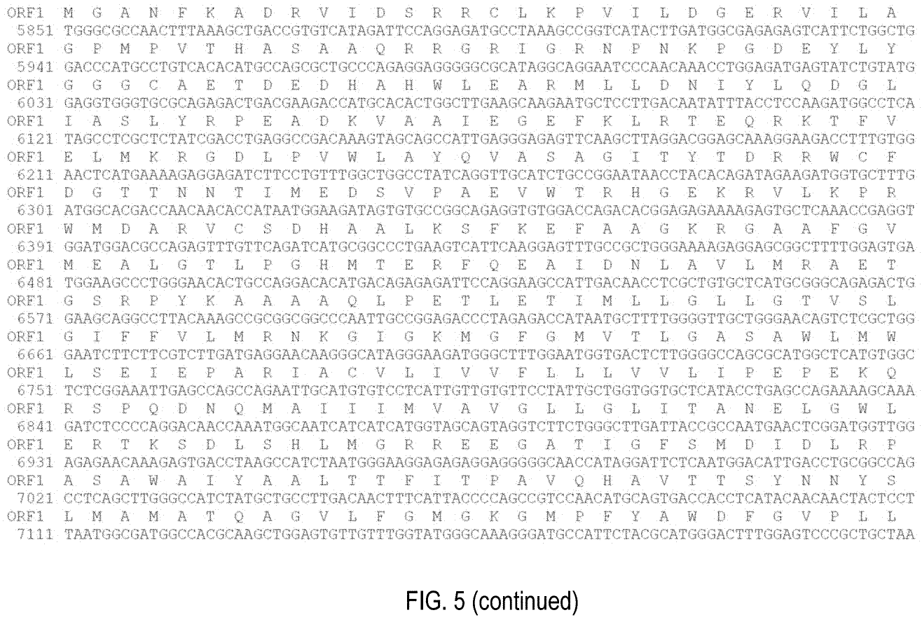

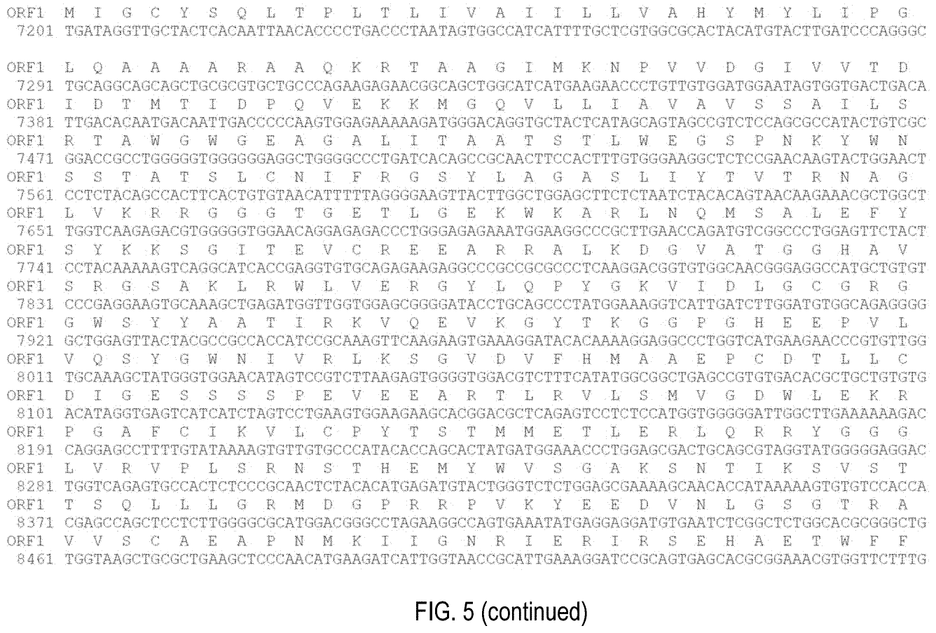

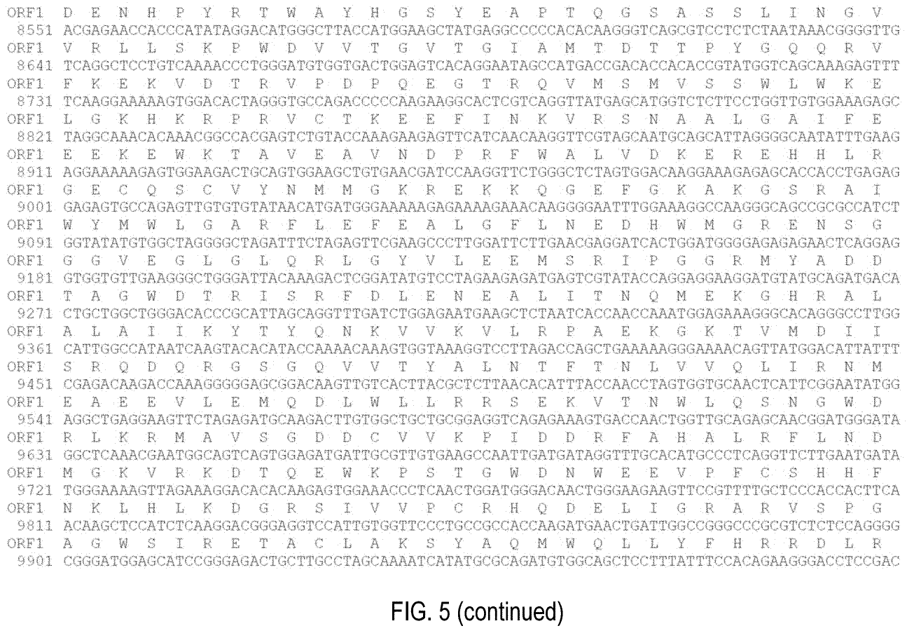

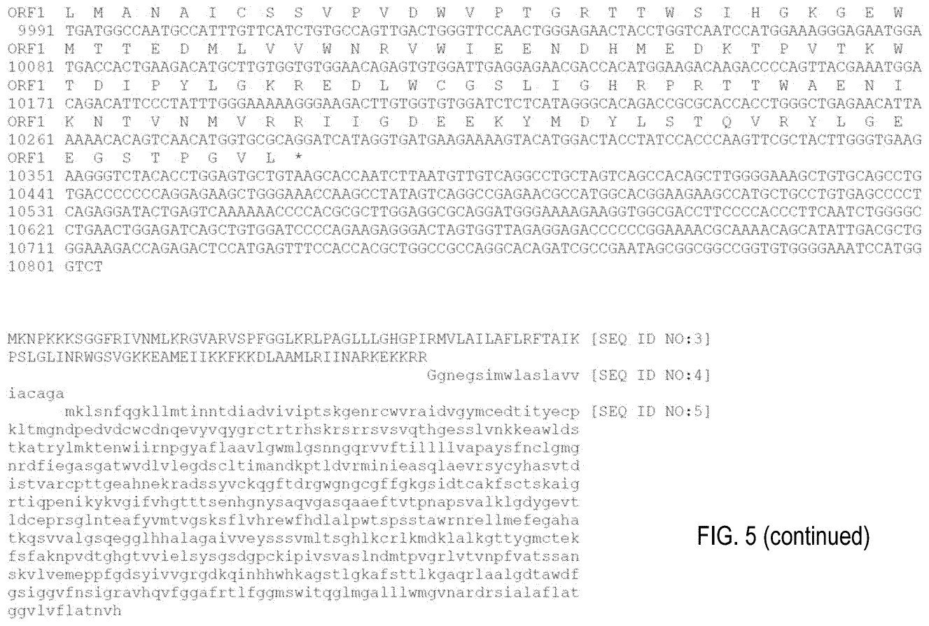

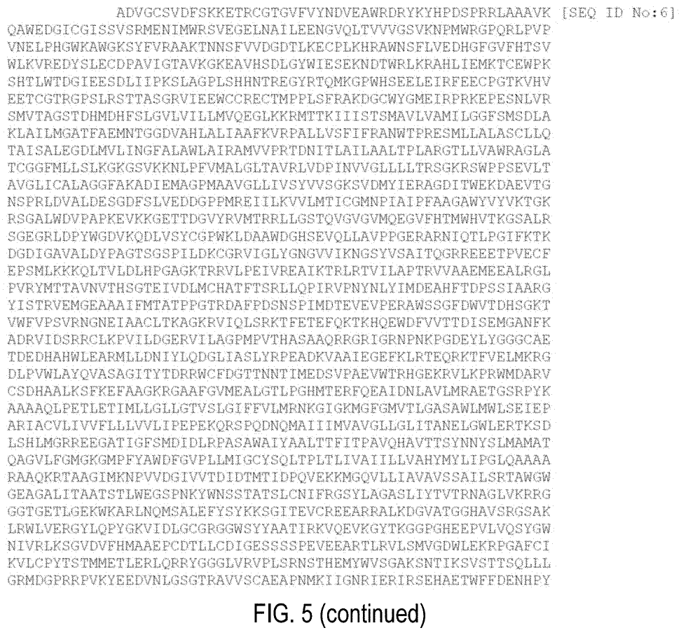

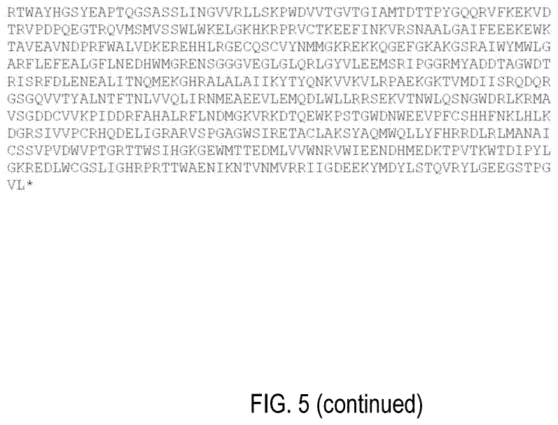

Transfection of both constructs in Vero E6 cells and unbiased monitoring of replication of the recombinant virus progeny by immune fluorescence microscopy revealed that unexpectedly the ZIKCanch variant failed to produce infectious progeny, by contrast to prior evidence from the construction of similar yellow fever virus ZIKV chimeras (Kum et al. (2018) NPJ Vaccines. 3, 56). Likewise, only the JECanch variant was fully replication competent (FIG. 2B). The thus generated virus ZIK-JEprM/E was proven to be a genetic chimera expressing the E protein of JEV and the NS1 protein of ZIKV (FIG. 2E). Further passaging of the chimeric ZIK-JEprM/E (JECanch variant) on Vero E6 cells for five passages lead to selection of an unpredictable adenin-to-guanosin nucleotide change A1482G at nucleotide position 1482 (A1482G) (see FIG. 5) resulting to a missense mutation in the ZIK-JEprM/E polyprotein, corresponding to a lysine-to-glutamate amino acid change in the JEV E-protein at position 166 (K166E).

TABLE 1
Correspondence between nucleotide
and amino acid substitution

| Nucleotide position in | |
|---|---|
| ZIK-JEprM/E JECanch: | A1482G |
| ZIK-JEprM/E JECanch | K459E |
| polyprotein | |
| JEV SA14-14-2 prM/E protein | K333E |
| JEV SA14-14-2 E protein | K166E |

The resulting ZIK-JEprM/E chimeric virus was attenuated as compared to the parental ZIKV, demonstrated by reduced replication kinetics on Vero E6 cells (FIG. 2C) and a smaller plaque size on BHK-21J cells (FIG. 2D).

### Example 2

### Attenuation, Immunogenicity and Vaccine Efficacy of ZIK-JEprM/E

The ZIK-JEprM/E vaccine virus was well tolerated when inoculated intraperitoneally (i.p.) into interferon alpha/beta (IFN-α/β)-deficient (AG129) mice at a dose of 10⁴ PFU (plaque forming units) of ZIK-JEprM/E; mice inoculated with a similar or even much lower dose (<10² PFU) of wild-type ZIKV uniformly die from infection again proving the favorable attenuation of the ZIK-JEprM/E vaccine virus.

Virus-specific neutralizing antibodies (nAb) are regularly elicited against the respective E protein present in or expressed by a particular flavivirus vaccine. High titres of nAb against a specific flavivirus are generally accepted to confer protection from infection with that specific virus. In lack of ZIKV E sequences ZIK-JEprM/E can therefore not induce other than JEV specific nAb. In line, AG129 vaccinated with ZIK-JEprM/E developed high titers of anti-JEV neutralizing antibodies (nAb), yet no nAb against ZIKV.

However, antibodies that are not neutralising, yet bind other proteins of ZIKV (non-nAb most likely directed against the ZIKV NS1 protein) were readily detectable in ZIK-JEprM/E AG129 mice by an indirect immune fluorescence assay. AG129 vaccinated with ZIK-JEprM/E also showed the strong induction of cell mediated immunity against the ZIKV, as demonstrated by a high number of ZIKV-specific IFN-γ secreting splenocytes as detected by ELISPOT ten weeks post vaccination with ZIK-JEprM/E (FIG. 3).

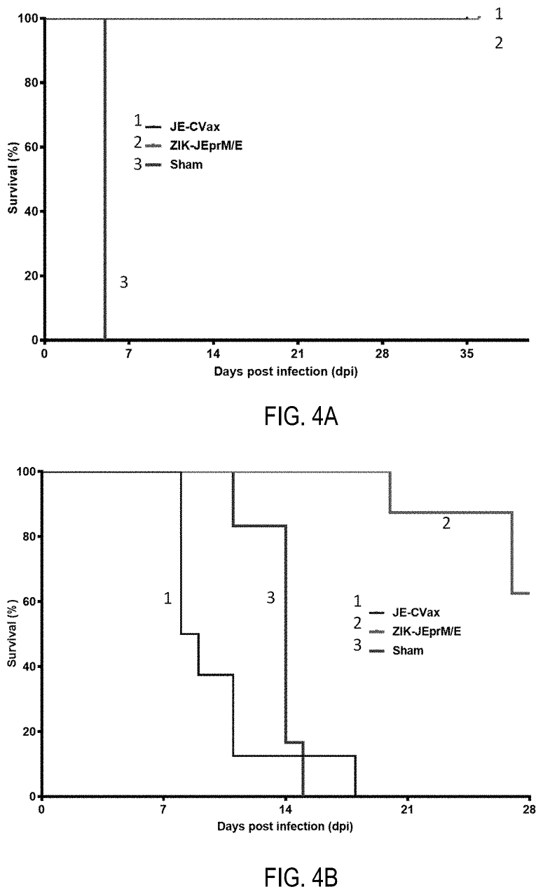

ZIK-JEprM/E vaccinated AG129 mice survived a lethal challenge with JEV (FIG. 4A) similarly to mice vaccinated with a YFV-17D based live-attenuated JEV (Chimerivax-JE; Imojev®) proving its efficacy to protect from a vigorous uniformly lethal JEV infection.

In contrast to the licensed JEV vaccine Chimerivax-JE, ZIK-JEprM/E also protected against a subsequent lethal ZIKV infection (FIG. 4B) conferring 60% survival in this very stringent challenge model, showing that ZIK-JEprM/E is also an efficient ZIKV vaccine. This protection by ZIK-JEprM/E against ZIKV is fully unexpected because (i) protection against flaviviruses is considered to be based on the specific induction of nAb that cannot be induced by ZIK-JEprM/E.

1-29. (canceled)

30. A polynucleotide comprising a nucleotide sequence of a live, infectious, attenuated Zika-Japanese encephalitis chimeric virus, wherein the nucleotide sequence encodes an amino acid sequence of a prME protein of a Japanese encephalitis virus, so that the prME protein of the Japanese encephalitis virus is expressed.

31. The polynucleotide according to claim 30, wherein the nucleotide sequence encodes an amino acid sequence of a signal sequence of a C terminal part of a C protein of the Japanese encephalitis virus so that the signal sequence of the Japanese encephalitis virus is expressed.

32. The polynucleotide according to claim 30, wherein the nucleotide sequence differs compared to the wild type sequence of the Japanese encephalitis virus E gene region depicted in SEQ ID NO:1 by an A to G nucleotide substitution at position 1482.

33. The polynucleotide according to claim 30, wherein the encoded amino acid sequence of the prME protein of the Japanese encephalitis virus has the sequence of SEQ ID NO:5.

34. The polynucleotide according to claim 30, wherein the nucleotide sequence encodes an amino acid sequence of a signal sequence of a C terminal part of a C protein of the Japanese encephalitis virus, and wherein the encoded amino acid sequence of the signal sequence of the C terminal part of the C protein of the Japanese encephalitis virus has the sequence GGNEGSIMWLASLAVV (SEQ ID NO:4).

35. The polynucleotide according to claim 30, wherein the nucleotide sequence encodes an amino acid sequence linking the Zika C protein and the signal sequence of the C terminal part of the C protein of the Japanese encephalitis virus, the encoded amino acid sequence linking the Zika C protein and the signal sequence of the C terminal part of the C protein of the Japanese encephalitis virus comprising a sequence EKKRR GGNEG (SEQ ID NO:7).

36. The polynucleotide according to claim 35, wherein:
the nucleotide sequence encodes an amino acid sequence linking the Zika C protein and the signal sequence of the C terminal part of the C protein of the Japanese encephalitis virus, the encoded amino acid sequence linking the Zika C protein and the signal sequence of the C terminal part of the C protein of the Japanese encephalitis virus comprising a sequence EKKRR GGNEG (SEQ ID NO:7); and/or
the nucleotide sequence encodes an amino acid sequence linking the prME protein of the Japanese encephalitis virus and the Zika NS protein, the encoded amino acid sequence linking the prME protein of the Japanese encephalitis virus and the Zika NS protein comprising the sequence ATNVH ADVGC (SEQ ID NO:8).

37. The polynucleotide according to claim 30, wherein the Zika virus is the BeH819015 strain.

38. The polynucleotide according to claim 30, comprising the open reading frame from nucleotide 108 to 10379 depicted in SEQ ID NO:1 with stopcodon included.

39. The polynucleotide according to claim 30, comprising the nucleotide sequence depicted in SEQ ID NO:1.

40. The polynucleotide according to claim 30, wherein the nucleotide sequence encodes the polypeptide sequence depicted in SEQ ID NO:2.

41. The polynucleotide according to claim 30, which is a Bacterial Artificial Chromosome.

42. The polynucleotide according to claim 41, wherein the Bacterial Artificial Chromosome comprises:
an inducible bacterial ori sequence for amplification of the Bacterial Artificial Chromosome to more than 10 copies per bacterial cell; and
a viral expression cassette comprising a cDNA of the Zika-Japanese encephalitis chimeric virus and comprising cis-regulatory elements for transcription of the viral cDNA in mammalian cells and for processing of the transcribed RNA into infectious RNA virus.

43. A live, infectious, attenuated Zika-Japanese encephalitis chimeric virus, wherein a prME protein of the chimeric virus is the prME protein of a Japanese encephalitis virus.

44. The Zika-Japanese encephalitis chimeric virus according to claim 43, wherein a signal sequence of the C terminal part of the C protein of the chimeric virus is the signal sequence of the C terminal part of the C protein of a Japanese encephalitis virus.

45. The Zika-Japanese encephalitis chimeric virus according to claim 43, comprising in a prME polypeptide the sequence QAAEFTV (SEQ ID NO:9).

46. The Zika-Japanese encephalitis chimeric virus according to claim 43, wherein the signal sequence and the prME protein of the Japanese encephalitis virus has the amino acid sequence depicted in SEQ ID NO:4 and SEQ ID NO:5.

47. The Zika-Japanese encephalitis chimeric virus according to claim 43, wherein the Japanese encephalitis virus is SA14-14-2.

48. The Zika-Japanese encephalitis chimeric virus according to claim 43, wherein the Zika virus is the BeH819015 strain.

49. The Zika-Japanese encephalitis chimeric virus according to claim 43, comprising an amino acid sequence GGNEGSIMWLASLAVV (SEQ ID NO:4), and comprising at a junction of the C protein of the Zika virus and a signal peptide of the Japanese encephalitis virus an amino acid sequence EKKRR GGNEG (SEQ ID NO:7).

50. A pharmaceutical composition comprising:
a polynucleotide sequence encoding a Zika-Japanese encephalitis chimeric virus according to claim 30; and
a pharmaceutical acceptable carrier.

51. A pharmaceutical composition comprising:
a Zika-Japanese encephalitis chimeric virus according to claim 43; and
a pharmaceutical acceptable carrier.

52. A method of preparing a vaccine against a Zika and/or Japanese encephalitis infection, the method comprising:
(a) providing a Bacterial Artificial Chromosome, the Bacterial Artificial Chromosome comprising:
an inducible bacterial ori sequence for amplification of the Bacterial Artificial Chromosome to more than 10 copies per bacterial cell; and
a viral expression cassette comprising:
a cDNA of a Zika-Japanese encephalitis chimeric virus according to claim 30; and
cis-regulatory elements for transcription of the viral cDNA in mammalian cells and for processing of the transcribed RNA into infectious RNA virus;
(b) transfecting mammalian cells with the Bacterial Artificial Chromosome of (a) and passaging infected cells;
(c) validating replicated virus of the transfected cells of (b) for virulence and the capacity of generating antibodies and inducing protection against Zika virus infection;
(d) cloning the virus validated in (c) into a vector; and
(e) formulating the vector into a vaccine formulation.

53. The method according to claim 52, wherein the vector is a Bacterial Artificial Chromosome which comprises an inducible bacterial ori sequence for amplification of the Bacterial Artificial Chromosome to more than 10 copies per bacterial cell.

54. A method of inducing a neutralizing and/or protective antibody response against Japanese encephalitis virus and/or Zika virus in a subject, thereby preventing an infection by Japanese encephalitis virus and/or Zika, the method comprising:
administering to the subject the Zika-Japanese encephalitis chimeric virus of claim 43.

55. A method of inducing a protective immune response against Japanese encephalitis virus and/or Zika virus in a subject, thereby preventing an infection by Japanese encephalitis virus and/or Zika, the method comprising:
administering to the subject the Zika-Japanese encephalitis chimeric virus of claim 43.
